(19) United States
(12) Patent Application Publication
(10) Pub. No.: US 2011/0062343 A1
(43) Pub. Date: Mar. 17, 2011

(54) Radiation imaging apparatus

(75) Inventor: Kenichi Kato, Ashigarakami-gun (JP)

(73) Assignee: FUJIFILM Corporation, Tokyo (JP)

(21) Appl. No.: 12/923,112

(22) Filed: Sep. 2, 2010

(30) Foreign Application Priority Data
Sep. 16, 2009 (JP) 2009-214125

(51) Int. Cl. A61B 6/00

(52) U.S. Cl. 250/393

(57) ABSTRACT

A radiation imaging operation, wherein a radiation source irradiates radiation to an object, while the radiation source is being moved in one direction, is performed a plurality of times with the radiation source taking various different positions. A radiation detector is reciprocally moved within a period, during which the radiation source is moved in the one direction from a stage of beginning of the plurality of times of the radiation imaging operations to a stage of finishing of the plurality of times of the radiation imaging operations. The radiation detector is moved in a direction opposite to the one direction of the movement of the radiation source within each of radiation imaging operation periods.

### BACKGROUND OF THE INVENTION

1. Field of the Invention

This invention relates to a radiation imaging apparatus for performing a plurality of times of radiation imaging operations on an object from a plurality of different angles as in the cases of tomosynthesis imaging operations, and the like.

2. Description of the Related Art

Heretofore, tomosynthesis imaging operations have been performed, wherein a plurality of radiation images of an object are acquired by performing a plurality of times of radiation imaging operations on the object from a plurality of different angles, and wherein a tomography image of an arbitrary height is reconstructed from the plurality of the radiation image having thus been acquired. (Reference may be made to, for example, U.S. Pat. No. 6,970,531.) U.S. Pat. No. 6,970,531 discloses a technique, wherein tomosynthesis imaging operations are performed, while a radiation source is being moved with respect to an object, and wherein irradiation of radiation is performed a plurality of times with a radiation detector being secured at a predetermined position. U.S. Pat. No. 6,970,531 also discloses a technique, wherein tomosynthesis imaging operations are performed, while a radiation source is being moved with respect to an object, and wherein a radiation detector is moved with respect to the radiation source in accordance with the linear movement of the radiation source.

However, in cases where the radiation detector is moved with respect to the radiation source in accordance with the movement of the radiation source, since the radiation detector continues to move in a direction opposite to the direction of the movement of the radiation source, the movement range of the radiation detector becomes large, and the problems occur in that the size of the imaging apparatus is not capable of being kept small. In cases where the radiation detector is secured at the predetermined position, since the irradiation of the radiation is performed, while the radiation source is moving, the so-called panning state arises, and the problems occur in that image quality of the acquired radiation image becomes bad.

### SUMMARY OF THE INVENTION

The primary object of the present invention is to provide a radiation imaging apparatus, wherein image quality of an acquired radiation image is prevented from becoming bad, and wherein an apparatus size is kept small.

Another object of the present invention is to provide a radiation imaging apparatus, wherein control of driving of a radiation source and the control of the driving of a radiation detector are performed efficiently.

A further object of the present invention is to provide a radiation imaging apparatus, wherein variation in image quality among a plurality of acquired radiation images is suppressed.

A still further object of the present invention is to provide a radiation imaging apparatus, wherein a radiation detector is reciprocally moved in a well-balanced manner.

The present invention provides a radiation imaging apparatus, comprising:

i) a radiation source for irradiating radiation to an object,

ii) a radiation detector for detecting the radiation, which has been irradiated from the radiation source to the object, and which carries image information of the object, as a radiation image, iii) radiation source moving means for linearly moving the radiation source along a predetermined direction with respect to the object,

iv) radiation detector moving means for moving the radiation detector in parallel with the direction of the movement of the radiation source, and

v) imaging operation control means for controlling such that a radiation imaging operation, wherein the radiation source irradiates the radiation to the object, while the radiation source is being moved in one direction, is performed a plurality of times with the radiation source taking various different positions,

the imaging operation control means controlling the radiation detector moving means such that the radiation detector is reciprocally moved within a period, during which the radiation source is moved in the one direction from a stage of beginning of the plurality of times of the radiation imaging operations to a stage of finishing of the plurality of times of the radiation imaging operations, and such that the radiation detector is moved in a direction opposite to the one direction of the movement of the radiation source within each of radiation imaging operation periods.

The radiation imaging apparatus in accordance with the present invention may be, for example, a radiation imaging apparatus for performing chest imaging operations. Alternatively, the radiation imaging apparatus in accordance with the present invention may be a mammography apparatus for performing mamma imaging operations. Also, it is sufficient for the radiation imaging apparatus in accordance with the present invention to perform the plurality of times of the radiation imaging operations, while the radiation source is being moved. Thus the radiation imaging apparatus in accordance with the present invention may be an apparatus for performing tomosynthesis imaging operations. Alternatively, the radiation imaging apparatus in accordance with the present invention may be an apparatus for performing long size imaging operations. In cases where the radiation imaging apparatus in accordance with the present invention is the apparatus for performing the tomosynthesis imaging operations, the radiation imaging apparatus in accordance with the present invention may further comprise image processing means for forming an image of a predetermined objective plane of the object by use of the plurality of the radiation images having been detected by the radiation detector.

Also, it is sufficient for the radiation source and the radiation detector to be moved in parallel with each other. The radiation source and the radiation detector may be moved in parallel with a direction of a body axis of a patient. Alternatively, the radiation source and the radiation detector may be moved in parallel with a direction, which intersects approximately perpendicularly to the body axis of the patient.

Further, the imaging operation control means controls the radiation detector moving means such that the radiation detector is moved in the direction opposite to the direction of the movement of the radiation source at the time of each radiation imaging operation. The radiation imaging apparatus in accordance with the present invention should preferably be modified such that the imaging operation control means controls such that a movement velocity of the radiation source is approximately identical among the plurality of the radiation imaging operation periods, and such that the movement velocity of the radiation detector is approximately identical among the plurality of the radiation imaging operation periods.

Furthermore, the imaging operation control means may control the movement of the radiation detector at the time of each of the radiation imaging operations. Alternatively, the radiation imaging apparatus in accordance with the present invention may be modified such that the imaging operation control means controls such that the radiation imaging operations are performed at predetermined imaging operation intervals, such that the radiation source is moved approximately at a predetermined velocity from an imaging operation beginning position to an imaging operation finishing position, and such that the radiation detector is reciprocally moved approximately with a predetermined period, and

the imaging operation control means controls in accordance with the movement velocity of the radiation source and the period, with which the radiation detector is reciprocally moved, such that the radiation source and the radiation detector are moved in opposite directions within each of the radiation imaging operation periods.

In such cases, the radiation imaging apparatus in accordance with the present invention may further be modified such that the imaging operation control means adjusts a timing, with which the driving of the radiation source moving means or the radiation detector moving means is begun, such that the radiation source and the radiation detector are moved in the opposite directions at the time of a first radiation imaging operation.

Also, it is sufficient for the radiation detector moving means to reciprocally move the radiation detector. The radiation detector moving means may reciprocally move the radiation detector such that the movement velocity at the time of the radiation imaging operation and the movement velocity at the time other than the radiation imaging operation are different from each other. Alternatively, the radiation detector moving means may reciprocally move the radiation detector with a predetermined period. In such cases, the radiation imaging apparatus in accordance with the present invention may be modified such that the radiation detector moving means is provided with driving means for reciprocally moving the radiation detector with a predetermined period, and

a balancing weight member, which is associated with the driving means, and which is reciprocally moved in a direction opposite to the direction of the movement of the radiation detector.

Further, the radiation imaging apparatus in accordance with the present invention may be modified such that the imaging operation control means sets a distance of the movement of the radiation detector within each of the radiation imaging operation periods in accordance with the distance of the movement of the radiation source within each of the radiation imaging operation periods. For example, the radiation imaging apparatus in accordance with the present invention may be modified such that the imaging operation control means calculates a blur quantity on the radiation detector from a distance of the movement of the radiation source within each of the radiation imaging operation periods and moves the radiation detector within each of the radiation imaging operation periods by a distance corresponding to the calculated blur quantity.

The radiation imaging apparatus in accordance with the present invention comprises:

i) the radiation source for irradiating the radiation to the object,

ii) the radiation detector for detecting the radiation, which has been irradiated from the radiation source to the object, and which carries the image information of the object, as the radiation image,

iii) the radiation source moving means for linearly moving the radiation source along the predetermined direction with respect to the object,

iv) the radiation detector moving means for moving the radiation detector in parallel with the direction of the movement of the radiation source, and

v) the imaging operation control means for controlling such that the radiation imaging operation, wherein the radiation source irradiates the radiation to the object, while the radiation source is being moved in one direction, is performed the plurality of times with the radiation source taking various different positions,

the imaging operation control means controlling the radiation detector moving means such that the radiation detector is reciprocally moved within the period, during which the radiation source is moved in the one direction from the stage of the beginning of the plurality of times of the radiation imaging operations to the stage of the finishing of the plurality of times of the radiation imaging operations, and such that the radiation detector is moved in the direction opposite to the one direction of the movement of the radiation source within each of the radiation imaging operation periods.

With the radiation imaging apparatus in accordance with the present invention, wherein the radiation imaging operation is performed, while the radiation detector is being moved in the direction opposite to the direction of the movement of the radiation source, the image quality of the acquired radiation image is prevented from becoming bad due to the movement of the radiation source. Also, since only the space for the reciprocal movement of the radiation detector may be prepared, the movement range of the radiation detector is suppressed to the minimum, and the apparatus size is kept small.

The radiation imaging apparatus in accordance with the present invention may be modified such that the imaging operation control means controls such that the radiation imaging operations are performed at the predetermined imaging operation intervals, such that the radiation source is moved approximately at the predetermined velocity from the imaging operation beginning position to the imaging operation finishing position, and such that the radiation detector is reciprocally moved approximately with the predetermined period, and

the imaging operation control means adjusts the timing, with which the driving of the radiation source moving means or the radiation detector moving means is begun, such that the radiation source and the radiation detector are moved in the opposite directions at the time of the first radiation imaging operation.

With the modification described above, it is not necessary to perform the control for altering the operations of the radiation source moving means and the radiation detector moving means for each of the radiation imaging operations, and the control of the driving of the radiation source and the control of the driving of the radiation detector are performed efficiently.

The radiation imaging apparatus in accordance with the present invention should preferably be modified such that the imaging operation control means controls such that the movement velocity of the radiation source is approximately identical among the plurality of the radiation imaging operation periods, and such that the movement velocity of the radiation detector is approximately identical among the plurality of the radiation imaging operation periods. With the modification described above, variation in image quality among the plurality of the acquired radiation images, which variation arises due to a difference in movement velocity, is suppressed.

Also, the radiation imaging apparatus in accordance with the present invention may be modified such that the radiation detector moving means is provided with the driving means for reciprocally moving the radiation detector with the predetermined period, and

the balancing weight member, which is associated with the driving means and which is reciprocally moved in the direction opposite to the direction of the movement of the radiation detector.

With the modification described above, the radiation detector is reciprocally moved in a well-balanced manner.

Further, the radiation imaging apparatus in accordance with the present invention may be modified such that the imaging operation control means sets the distance of the movement of the radiation detector within each of the radiation imaging operation periods in accordance with the distance of the movement of the radiation source within each of the radiation imaging operation periods. Particularly, the radiation imaging apparatus in accordance with the present invention may be modified such that the imaging operation control means calculates the blur quantity on the radiation detector from the distance of the movement of the radiation source within each of the radiation imaging operation periods and moves the radiation detector within each of the radiation imaging operation periods by the distance corresponding to the calculated blur quantity. With the modification described above, the image quality of the acquired radiation image is reliably prevented from becoming bad due to the radiation imaging operation, which is performed, while the radiation source is being moved.

Furthermore, the radiation imaging apparatus in accordance with the present invention may be modified such that, at the time at which the plurality of times of the radiation imaging operations are performed on a patient acting as the object, the radiation source and the radiation detector are moved in parallel with the direction, which intersects approximately perpendicularly to the body axis of the patient. With the modification described above, a radiation dose delivered to the patient is kept small.

### BRIEF DESCRIPTION OF THE DRAWINGS

FIG. 1 is a schematic view showing an embodiment of the radiation imaging apparatus in accordance with the present invention,

FIG. 2 is an explanatory view showing an example of radiation detector moving means in the radiation imaging apparatus of FIG. 1,

FIG. 3 is a graph showing how a position of a radiation detector is altered with the passage of time by the radiation detector moving means of FIG. 2,

FIG. 4 is a flow chart showing an example of an operation of the radiation imaging apparatus in accordance with the present invention,

FIG. 5 is an explanatory view showing a different example of the radiation detector moving means in the radiation imaging apparatus in accordance with the present invention,

FIG. 6 is an explanatory view showing a further different example of the radiation detector moving means in the radiation imaging apparatus in accordance with the present invention,

FIG. 7 is an explanatory view showing a still further different example of the radiation detector moving means in the radiation imaging apparatus in accordance with the present invention,

FIG. 8 is a graph showing how the radiation detector is reciprocally moved by the radiation detector moving means, and

FIG. 9 is a schematic view showing a different embodiment of the radiation imaging apparatus in accordance with the present invention.

### DESCRIPTION OF THE PREFERRED EMBODIMENTS

The present invention will hereinbelow be described in further detail with reference to the accompanying drawings.

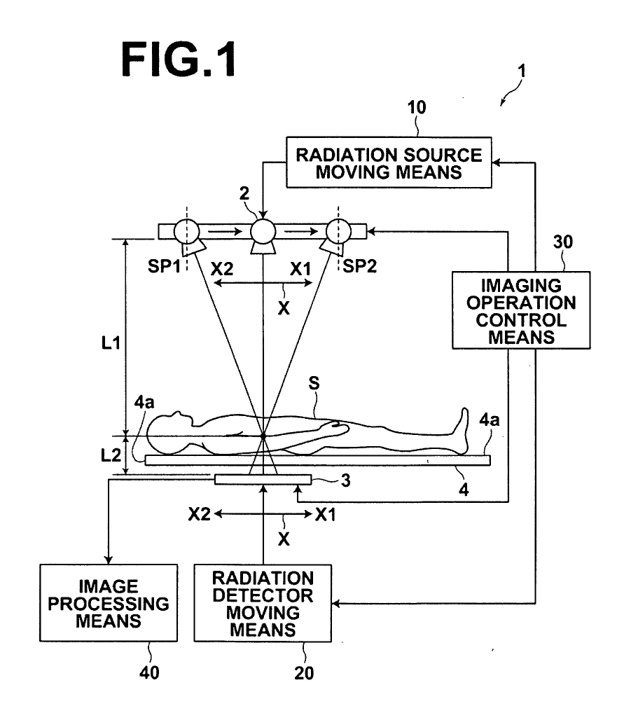

FIG. 1 is a schematic view showing an embodiment of the radiation imaging apparatus in accordance with the present invention. With reference to FIG. 1, a radiation imaging apparatus 1 is constructed for performing tomosynthesis imaging operations on the chest, the abdomen, and the like. The radiation imaging apparatus 1 comprises a radiation source 2, a radiation detector 3, radiation source moving means 10, radiation detector moving means 20, imaging operation control means 30, and image processing means 40.

The radiation source 2 irradiates radiation to an object. The radiation source 2 is capable of being moved linearly along a predetermined direction (along the direction indicated by the double headed arrow X, i.e. along the direction of a body axis of an object S) with respect to the object S by the radiation source moving means 10. Particularly, in cases where the tomosynthesis imaging operations are performed, the radiation source moving means 10 moves the radiation source 2 toward the direction indicated by the arrow X1 over a predetermined distance (e.g., 1.2 m) from an imaging operation beginning position SP1 to an imaging operation finishing position SP2. The radiation source 2 irradiates the radiation to the object S, while the radiation source 2 is being moved in the direction indicated by the arrow X1. A plurality of times of radiation imaging operations are performed within the period, during which the radiation source 2 is moved from the imaging operation beginning position SP1 to the imaging operation finishing position SP2. The radiation source moving means 10 has the function for adjusting the angle of the radiation, which is irradiated from the radiation source 2, in accordance with the movement of the radiation source 2, such that the radiation is irradiated to a region of interest of the object S.

The radiation detector 3 detects the radiation, which has been irradiated from the radiation source 2 to the object S, and which carries the image information of the object S, as a radiation image. As the radiation detector 3, it is possible to employ the so-called TFT type detector or an optical readout type detector. The image processing means 40 forms a tomosynthesis image of a predetermined objective plane of the object S in accordance with the plurality of the radiation images having been detected by the radiation detector 3. The radiation detector 3 is connected with the radiation detector moving means 20 for reciprocally moving the radiation detector 3 in parallel with the direction of the movement of the radiation source 2 (i.e., the direction indicated by the double headed arrow X). The radiation detector moving means 20 is controlled by the imaging operation control means 30, such that the radiation detector moving means 20 reciprocally moves the radiation detector 3 in parallel with the direction indicated by the double headed arrow X within the period, during which the radiation source 2 is moved from the imaging operation beginning position SP1 to the imaging operation finishing position SP2.

The imaging operation control means 30 has the function for controlling the operations of the radiation source 2 and the radiation detector 3 at the time of each of the radiation imaging operations. The imaging operation control means 30 also has the function for controlling the operations of the radiation source moving means 10 and the radiation detector moving means 20. Specifically, the imaging operation control means 30 controls the radiation source 2 and the radiation detector 3 such that the radiation imaging operation, wherein the radiation source 2 irradiates the radiation to the object S, while the radiation source 2 is being moved in one direction (i.e., in the direction indicated by the arrow X1), is performed the plurality of times with the radiation source 2 taking various different positions. Also, the imaging operation control means 30 controls the radiation detector moving means 20 such that the radiation detector 3 is reciprocally moved within the period, during which the radiation source 2 is moved in the one direction (i.e., in the direction indicated by the arrow X1) from the stage of the beginning of the plurality of times of the radiation imaging operations to the stage of the finishing of the plurality of times of the radiation imaging operations, and such that the radiation detector 3 is moved in the direction (i.e., in the direction indicated by the arrow X2) opposite to the one direction of the movement of the radiation source 2 within each of radiation imaging operation periods RP, RP, . . . .

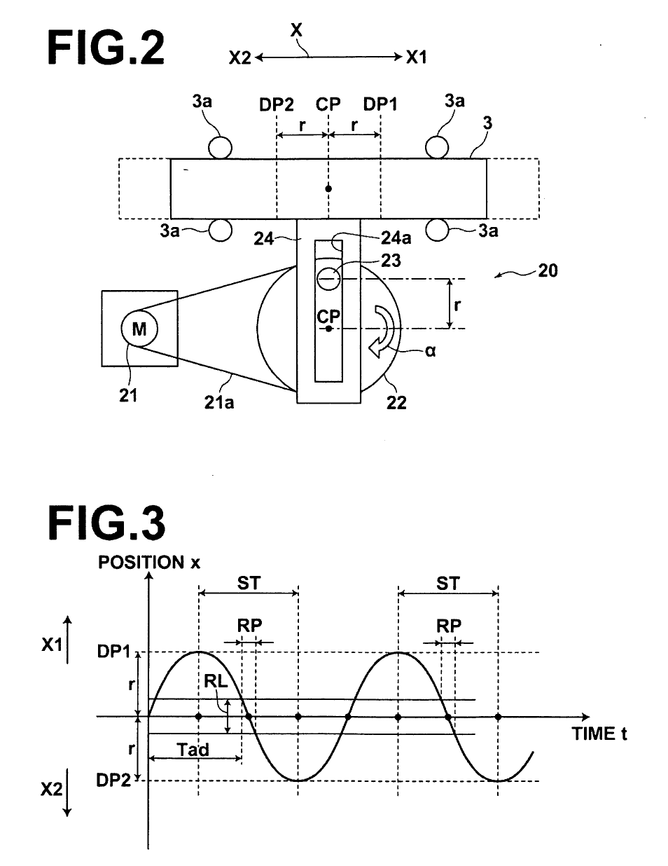

By way of example, the imaging operation control means 30 may move the radiation detector 3 at predetermined radiation imaging operation intervals, with a predetermined period, and with a predetermined amplitude. FIG. 2 is an explanatory view showing an example of radiation detector moving means in the radiation imaging apparatus of FIG. 1. Firstly, an example of the radiation detector moving means 20 adapted for moving the radiation detector 2 with a predetermined amplitude and with a predetermined period will be described hereinbelow with reference to FIG. 2. The radiation detector moving means 20 illustrated in FIG. 2 is provided with rotation driving means 21, a rotation member 22, a pin 23, and a slide member 24. The rotation driving means 21 is constituted of, for example, a motor, or the like. The operation of the rotation driving means 21 is controlled by the imaging operation control means 30. The rotation driving means 21 rotates the rotation member 22 via a belt 21a.

The rotation member 22 is constituted of a circular disk-shaped member, which is rotated by the rotation driving means 21 in the direction indicated by the arrow α with a center point CP acting as a rotation axis. A pin 23 is formed on a planar section of the rotation member 22. The pin 23 is associated with the slide member 24. The slide member 24 is constituted of a plate-like member, whose one side is secured to the radiation detector 3, and whose other side extends in the direction, which intersects perpendicularly to the detection surface of the radiation detector 3. A longitudinally extending rectangular hole 24a for insertion of the pin 23 is formed approximately at a center region of the slide member 24.

At the time at which the rotation driving means 21 is actuated, the rotation member 22 is rotated at a predetermined angular velocity in the direction indicated by the arrow α via the belt 21a, and the pin 23 formed on the rotation member 22 is rotated in the direction indicated by the arrow α. As a result, force is exerted from the pin 23 to the slide member 24, and the slide member 24 is reciprocally moved. In accordance with the reciprocal movement of the slide member 24, the radiation detector 3 is reciprocally moved along the direction indicated by the double headed arrow X. FIG. 3 is a graph showing how a position of a radiation detector is altered with the passage of time by the radiation detector moving means of FIG. 2. Specifically, as illustrated in FIG. 3, the radiation detector 3 is reciprocally moved with the predetermined amplitude and with the predetermined period between a position DP1 and a position DP2.

The period and the amplitude of the reciprocal movement of the radiation detector 3 are determined by a rotational velocity of the rotation member 22 and a radius r from the rotation center point CP to the pin 23. The rotational velocity of the rotation member 22 and a radius r are determined by the radiation imaging operation intervals and radiation imaging operation time (i.e., the irradiation time per radiation imaging operation). By way of example, it may be assumed that 30 radiation images are acquired under the conditions of the radiation imaging operation intervals of 200 ms and the radiation imaging operation time RP of 10 ms, while the radiation source 2 is being moved at uniform velocity in the direction indicated by the arrow X1. In such cases, the distance, by which the radiation source 2 is moved within the radiation imaging operation time RP, is equal to 2 mm (=1200/30/0.2*0.01). The movement of the radiation source 2 by the distance of 2 mm within the radiation imaging operation time RP causes blur to occur in the radiation image.

As illustrated in FIG. 1, it may be assumed that a distance L1 from the radiation source 2 to the object S (the region of interest) is equal to 1,000 mm, and that a distance L2 from the object S to the radiation detector 3 is equal to 100 mm. In such cases, the movement of the radiation source 2 by the distance of 2 mm causes a blur quantity of 0.2 mm (=2 mm*(L2/L1)) to occur in the radiation image. Therefore, in order for the blur to be canceled by the movement of the radiation detector 3, the radiation detector 3 may be moved in the direction indicated by the arrow X2 by the distance of 0.2 mm within the period of time of 10 ms, while the radiation source 2 is being moved in the direction indicated by the arrow X1 by the distance of 2 mm within the period of time of 10 ms.

Also, as illustrated in FIG. 3, the radiation imaging operation may be performed within a period ST, during which the radiation detector 3 is moved in the direction indicated by the arrow X2. It may be considered that each of the plurality of the radiation imaging operations is performed within a movement range such that the phase within the movement period of the radiation detector 3 is approximately identical. Specifically, it may be considered that each of the plurality of the radiation imaging operations is performed within the period, during which the radiation detector 3 is moved by a distance RL around a reference position of the reciprocal movement at the time of each of the radiation imaging operation intervals. In such cases, in order for the radiation imaging operation intervals of 200 ms and the radiation imaging operation time RP of 10 ms to be obtained, it is necessary that the rotation member 22 is rotated at an angular velocity of 18° (=360°/(10/200)) per 10 ms. At the time at which the rotation member 22 is rotated by the angle of 18°, the radiation detector 3 may be moved by the distance of 0.2 mm. Therefore, since a circular arc length=rθ, it is found that r=(18/180π)/0.2 mm=0.637 mm. Specifically, the pin 23 may be formed at the position 0.637 mm spaced from the rotation center point CP of the rotation member 22, and the rotation member 22 may be rotated at the angular velocity of 1.8°/ms. In such cases, the radiation detector 3 is moved by a distance of approximately 0.2 mm within the period of time of 10 ms at a velocity in the vicinity of the highest velocity with the timing matched with the irradiation at each of the intervals of 200 ms.

In cases where the positions of the radiation source 2 and the radiation detector 3 at the time of a first radiation imaging operation are reproduced also at the time of a second radiation imaging operation and those that follow as described above, the imaging operation control means 30 may control such that the radiation source 2 and the radiation detector 3 have a desired positional relationship at the time of the first radiation imaging operation. In such case, the driving control need not be performed independently for each of the second radiation imaging operation period RP and those that follow, and the control is thus performed efficiently. Therefore, the imaging operation control means 30 controls the timing, with which the driving of the radiation source moving means 10 is begun, and the timing, with which the driving of the radiation detector moving means 20 is begun, in accordance with the previously known factors, i.e., the intervals of the radiation imaging operation periods RP, RP, . . . , the initial position and the movement velocity of the radiation source 2, and the initial position and the movement velocity of the radiation detector 3.

Specifically, the imaging operation control means 30 calculates an initial radiation source movement time Tat, which is necessary for the radiation source 2 to arrive at the first imaging operation beginning position SP1 in an identical state in each radiation imaging operation, and an initial radiation detector movement time Tad, which is necessary for the radiation detector 3 to be located from a beginning position to the first imaging operation beginning position in the identical state in each radiation imaging operation. In cases where the initial radiation source movement time Tat is longer than the initial radiation detector movement time Tad (i.e., Tat>Tad), the radiation source moving means 10 begins the driving at a stage earlier by a length of time |Tat−Tad|than the radiation detector moving means 20. Also, in cases where the initial radiation source movement time Tat is shorter than the initial radiation detector movement time Tad (i.e., Tat<Tad), the radiation detector moving means 20 begins the driving at the stage earlier by the length of time |Tat−Tad| than the radiation source moving means 10. As a result, each of the second radiation imaging operation and those that follow is performed in the state identical with the state in the first radiation imaging operation, and variation in image quality among the plurality of the acquired radiation images is prevented from occurring due to a difference in movement distance of the radiation detector 3 among the plurality of times of the radiation imaging operations. In the embodiment described above, the imaging operation control means 30 adjusts both the timing, with which the driving of the radiation source moving means 10 is begun, and the timing, with which the driving of the radiation detector moving means 20 is begun. Alternatively, the imaging operation control means 30 may adjust only either one of the timing, with which the driving of the radiation source moving means 10 is begun, and the timing, with which the driving of the radiation detector moving means 20 is begun.

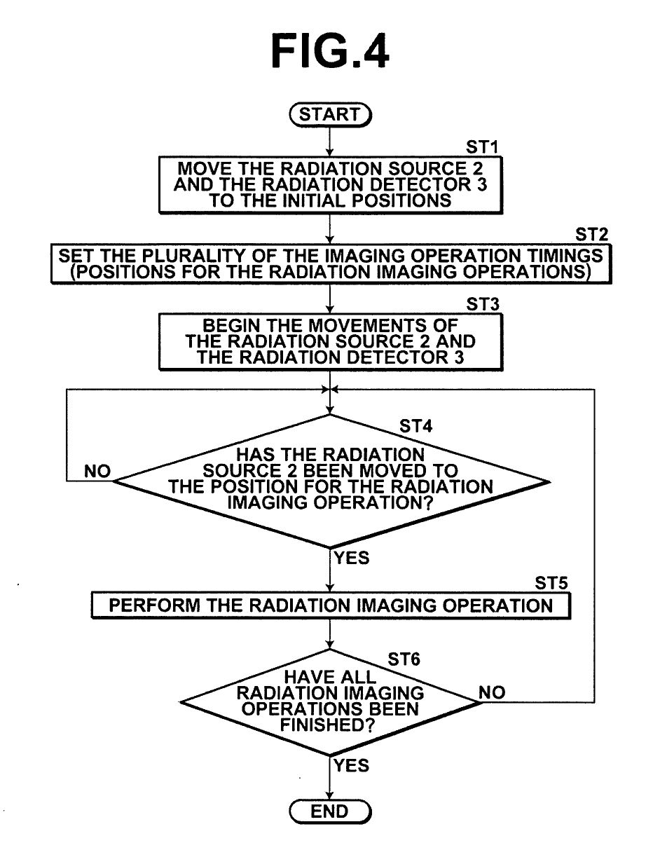

FIG. 4 is a flow chart showing an example of an operation of the radiation imaging apparatus in accordance with the present invention. An example of an operation of the radiation imaging apparatus in accordance with the present invention will be described hereinbelow with reference to FIG. 1 through FIG. 4. Firstly, before the first radiation imaging operation is begun, in a step ST1, the radiation source 2 and the radiation detector 3 are moved to the initial positions thereof. Also, in a step ST2, the imaging operation control means 30 sets the radiation imaging operation conditions, such as the position of the region of interest, the radiation imaging operation intervals, and the irradiation time. Thereafter, in a step ST3, the movement of the radiation source 2 in the direction indicated by the arrow X1 and the reciprocal movement of the radiation detector 3 are begun in accordance with the radiation imaging operation conditions. At this time, the imaging operation control means 30 adjusts the timing, with which the driving of the radiation source moving means 10 or the radiation detector moving means 20 is begun. The imaging operation control means 30 thus controls such that the first radiation imaging operation is performed in the predetermined state.

Further, in a step ST4 and a step ST5, at the time at which the radiation source 2 has been moved to the position for the first radiation imaging operation, the irradiation of the radiation from the radiation source 2 is begun, and the first radiation imaging operation is performed. At this time, as illustrated in FIG. 3, the radiation detector 3 is being moved within the radiation imaging operation period RP in the direction (indicated by the arrow X2) opposite to the direction of the movement of the radiation source 2. In a step ST6, the plurality of times of the radiation imaging operations are iterated, and the plurality of the radiation images are detected. Thereafter, the tomosynthesis processing is performed by the image processing means 40.

As described above, each of the radiation imaging operations is performed at the time, at which the radiation source 2 and the radiation detector 3 are being moved in opposite directions. The image quality of the acquired radiation image is thus prevented from becoming bad, and the apparatus size is kept small. Specifically, if the radiation detector 3 is set in a secured state as in a conventional technique in cases where the irradiation of the radiation from the radiation source 2 to the object S is performed, while the radiation source 2 is being moved, the image quality of the acquired radiation image will become bad. Also, if the radiation detector 3 continues to move in the direction opposite to the direction of the movement of the radiation source 2 as in a conventional technique in cases where the irradiation of the radiation from the radiation source 2 to the object S is performed, while the radiation source 2 is being moved, the size of the imaging apparatus is not capable of being kept small. With the embodiment of the radiation imaging apparatus in accordance with the present invention, wherein the radiation detector 3 is moved (vibrated) in the direction opposite to the direction of the movement of the radiation source 2 by the distance corresponding to the blur quantity, the image quality of the acquired radiation image is prevented from becoming bad, and the size of the radiation imaging apparatus is kept small.

Also, with the embodiment of the radiation imaging apparatus in accordance with the present invention, wherein the movement distance of the radiation detector 3 is short, it becomes possible for the tomosynthesis imaging operations to be performed with respect to every site of the object (patient) S lying on a bed 4 shown in FIG. 1. Specifically, in order for a radiation image of the region of interest of the object S to be obtained, it is necessary for the radiation, which has been irradiated to the object S and which carries the image information of the object S, to be projected onto the radiation detector 3. Therefore, if the radiation detector 3 continues to move in the direction opposite to the direction of the movement of the radiation source 2 as in the conventional technique, in cases where a site of the object S in the vicinity of an edge section 4a of the bed 4 is to be imaged, it will be necessary for the radiation detector 3 to be moved to a position spaced away from the edge section 4a of the bed 4. With the embodiment of the radiation imaging apparatus in accordance with the present invention, wherein the movement range of the radiation detector 3 is narrow and wherein the size of the radiation detector moving means 20 is kept small, it becomes possible for the tomosynthesis imaging operations to be performed even on the site of the object S in the vicinity of the edge section 4a of the bed 4, and serviceability of the radiation imaging apparatus is enhanced. In cases where the site of the object S in the vicinity of the edge section 4a of the bed 4 is to be imaged in FIG. 1, the reciprocal movement of the radiation detector 3 is performed after the radiation detector 3 has been moved to the position in the vicinity of the edge section 4a of the bed 4.

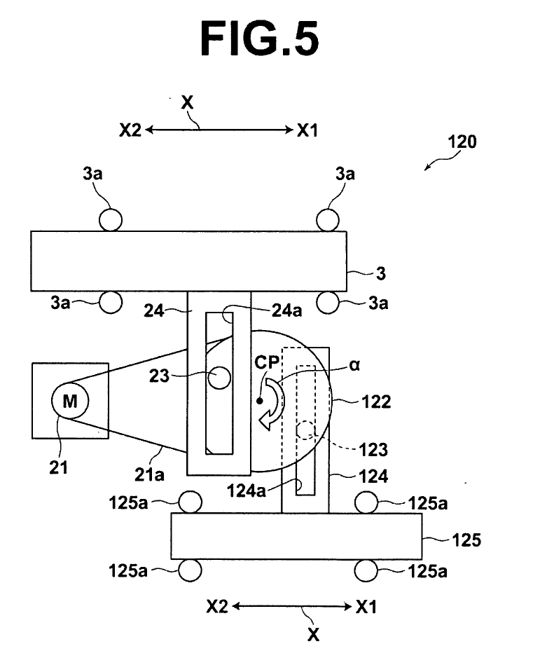

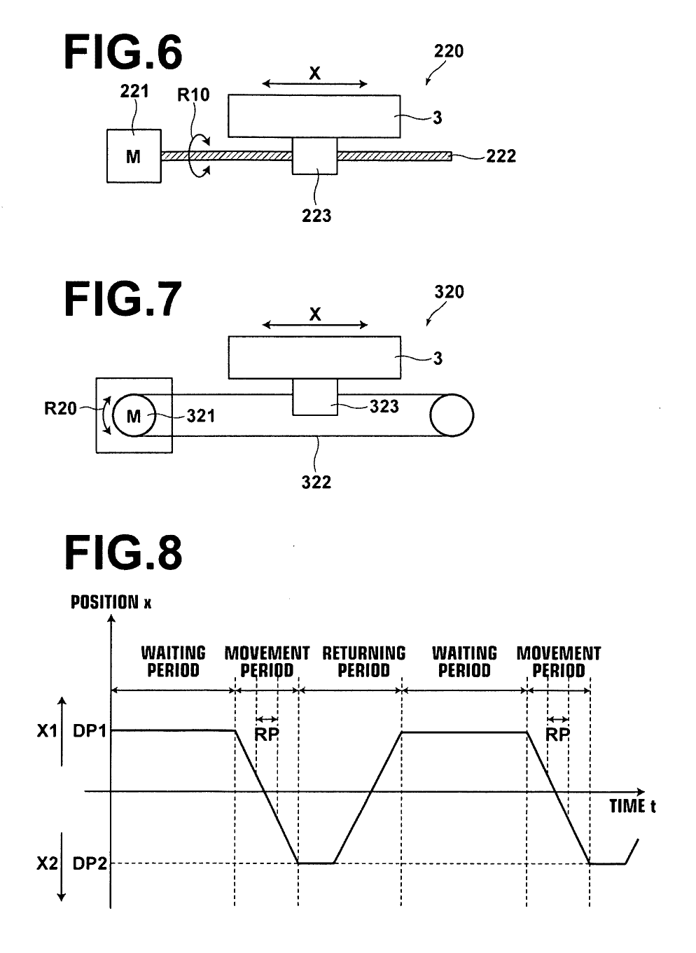

FIG. 5, FIG. 6, and FIG. 7 are explanatory views showing various different examples of the radiation detector moving means in the radiation imaging apparatus in accordance with the present invention. The various different examples of the radiation detector moving means will be described hereinbelow with reference to FIG. 5, FIG. 6, and FIG. 7. In FIG. 5, similar elements are numbered with the same reference numerals with respect to FIG. 2.

With reference to FIG. 5, radiation detector moving means 120 is constituted basically in the same manner as that for the radiation detector moving means 20 shown in FIG. 2, except that a balancing weight member 125 is associated with a rotation member 122, which is rotated by rotation driving means 121. Specifically, the first pin 23 is formed on one surface side of the rotation member 122, and a second pin 123 is formed on the other surface side of the rotation member 122. The second pin 123 is secured at a position varying in phase by 180° with respect to the position of the first pin 23 and is associated with a second slide member 124. The second slide member 124 has a shape extending approximately in parallel with the first slide member 24, and one edge side of the second slide member 124 is secured to the balancing weight member 125. A longitudinally extending rectangular hole 124a for insertion of the second pin 123 is formed approximately at a center region of the second slide member 124. At the time at which the radiation detector 3 is moved along the direction indicated by the double headed arrow X by the rotation of the rotation member 122, the balancing weight member 125 is moved along the direction indicated by the double headed arrow X toward the direction opposite to the direction of the movement of the radiation detector 3. In this manner, unnecessary vibration, or the like, does not arise with the radiation detector 3, and the stable reciprocal movement of the radiation detector 3 is performed.

A different example of the radiation detector moving means will be described hereinbelow with reference to FIG. 6. Radiation detector moving means 220 shown in FIG. 6 comprises rotation driving means 221, a ball screw 222, and a nut member 223. The rotation driving means 221 rotates the ball screw 222. By the driving of the rotation driving means 221, the ball screw 222 is rotated in the direction indicated by the double headed arrow R10 around a rotation axis. The nut member 223 is secured to the radiation detector 3 and is engaged with the ball screw 222. At the time at which the ball screw 222 is rotated along the direction indicated by the double headed arrow R10, the radiation detector 3 is moved along the direction indicated by the double headed arrow X.

A further different example of the radiation detector moving means will be described hereinbelow with reference to FIG. 7. Radiation detector moving means 320 shown in FIG. 7 comprises rotation driving means 321, a rotating belt 322, and a securing member 323. The rotation driving means 321 rotates the rotating belt 322 along the direction indicated by the double headed arrow R20. One side section of the rotating belt 322 is associated with the rotation driving means 321, and the other side section of the rotating belt 322 is supported for rotation by a roller. Also, the section of the rotating belt 322 between the rotation driving means 321 and the roller is kept in an approximately flat state. The securing member 323 is secured to the flat section of the rotating belt 322, and the radiation detector 3 is secured to the securing member 323. Therefore, at the time at which the rotating belt 322 is rotated by the rotation driving means 321, the radiation detector 3 is reciprocally moved along the direction indicated by the double headed arrow X.

In cases where the radiation detector moving means 220 shown in FIG. 6 or the radiation detector moving means 320 shown in FIG. 7 is employed, since the radiation detector 3 is moved toward the direction opposite to the direction of the movement of the radiation source 2 within each of the radiation imaging operation periods RP, RP, . . . , the image quality of the acquired radiation image is prevented from becoming bad, and the apparatus size is kept small.

The aforesaid embodiment of the radiation imaging apparatus in accordance with the present invention comprises:

i) the radiation source 2 for irradiating the radiation to the object S,

ii) the radiation detector 3 for detecting the radiation, which has been irradiated from the radiation source 2 to the object S, and which carries the image information of the object S, as the radiation image,

iii) the radiation source moving means 10 for linearly moving the radiation source 2 along the predetermined direction (i.e., the direction indicated by the double headed arrow X) with respect to the object S,

iv) the radiation detector moving means 20 for moving the radiation detector 3 in parallel with the direction of the movement of the radiation source 2 (i.e., the direction indicated by the double headed arrow X), and

v) the imaging operation control means 30 for controlling such that the radiation imaging operation, wherein the radiation source 2 irradiates the radiation to the object S, while the radiation source 2 is being moved in one direction (i.e., the direction indicated by the arrow X1), is performed the plurality of times with the radiation source 2 taking various different positions,

the imaging operation control means 30 controlling the radiation detector moving means 20 such that the radiation detector 3 is reciprocally moved within the period, during which the radiation source 2 is moved in the one direction (i.e., the direction indicated by the arrow X1) from the stage of the beginning of the plurality of times of the radiation imaging operations to the stage of the finishing of the plurality of times of the radiation imaging operations, and such that the radiation detector 3 is moved in the direction (i.e., the direction indicated by the arrow X2) opposite to the one direction ((i.e., the direction indicated by the arrow X1)) of the movement of the radiation source 2 within each of the radiation imaging operation periods.

With the aforesaid embodiment of the radiation imaging apparatus in accordance with the present invention, wherein the radiation imaging operation is performed, while the radiation detector 3 is being moved in the direction opposite to the direction of the movement of the radiation source 2, the image quality of the acquired radiation image is prevented from becoming bad due to the movement of the radiation source 2. Also, since only the space for the reciprocal movement of the radiation detector 3 may be prepared, the movement range of the radiation detector 3 is suppressed to the minimum, and the apparatus size is kept small.

Also, as illustrated in FIG. 2 and FIG. 3, the radiation imaging apparatus in accordance with the present invention may be modified such that the imaging operation control means 30 controls such that the radiation imaging operations are performed at the predetermined imaging operation intervals, such that the radiation source 2 is moved approximately at the predetermined velocity from the imaging operation beginning position SP1 to the imaging operation finishing position SP2, and such that the radiation detector 3 is reciprocally moved approximately with the predetermined period, and

the imaging operation control means 30 adjusts the timing, with which the driving of the radiation source moving means 10 or the radiation detector moving means 20 is begun, such that the radiation source 2 and the radiation detector 3 are moved in the opposite directions at the time of the first radiation imaging operation.

With the modification described above, it is not necessary to perform the control for altering the operations of the radiation source moving means 10 and the radiation detector moving means 20 for each of the radiation imaging operations, and the control of the driving of the radiation source 2 and the control of the driving of the radiation detector 3 are performed efficiently.

Further, the radiation imaging apparatus in accordance with the present invention should preferably be modified such that the imaging operation control means 30 controls such that the movement velocity of the radiation source 2 is approximately identical among the plurality of the radiation imaging operation periods RP, RP, . . . , and such that the movement velocity of the radiation detector 3 is approximately identical among the plurality of the radiation imaging operation periods RP, RP, . . . . With the modification described above, variation in image quality among the plurality of the acquired radiation images, which variation arises due to a difference in movement velocity, is suppressed.

Also, as illustrated in FIG. 5, the radiation imaging apparatus in accordance with the present invention may be modified such that the radiation detector moving means 120 is provided with the rotation driving means 121 for reciprocally moving the radiation detector 3 with the predetermined period, and

the balancing weight member 125, which is associated with the rotation driving means 121 and which is reciprocally moved in the direction opposite to the direction of the movement of the radiation detector 3.

With the modification described above, the radiation detector 3 is reciprocally moved in a well-balanced manner.

Further, the radiation imaging apparatus in accordance with the present invention may be modified such that the imaging operation control means 30 sets the distance of the movement of the radiation detector 3 within each of the radiation imaging operation periods RP, RP, . . . in accordance with the distance of the movement of the radiation source 2 within each of the radiation imaging operation periods RP, RP, . . . . Particularly, the radiation imaging apparatus in accordance with the present invention may be modified such that the imaging operation control means 30 calculates the blur quantity on the radiation detector 3 from the distance of the movement of the radiation source 2 within each of the radiation imaging operation periods RP, RP, . . . and moves the radiation detector 3 within each of the radiation imaging operation periods RP, RP, . . . by the distance corresponding to the calculated blur quantity. With the modification described above, the image quality of the acquired radiation image is reliably prevented from becoming bad due to the radiation imaging operation, which is performed, while the radiation source 2 is being moved.

The radiation imaging apparatus in accordance with the present invention may be embodied in various other ways. In the embodiment shown in FIG. 3, the radiation detector 3 iterates the reciprocal movement with the predetermined period and with the predetermined amplitude. Alternatively, as illustrated in FIG. 8, the imaging operation control means 30 may control such that the radiation detector 3 is moved at a predetermined velocity in the direction, which is indicated by the arrow X2, only in each of the radiation imaging operation periods RP, RP, . . . , such that the radiation detector 3 is returned in the direction, which is indicated by the arrow X1, at each stage after the radiation imaging operation has been finished, and such that the radiation detector 3 is then allowed to wait for the next radiation imaging operation.

Also, in the embodiment described above, the radiation imaging operations are performed at the predetermined imaging operation intervals. Alternatively, the radiation imaging operations may be performed at different imaging operation intervals. In such cases, the imaging operation control means 30 controls such that the radiation source 2 is moved, while the radiation source 2 is irradiating the radiation to the object S during each of the radiation imaging operations, and such that the radiation detector 3 is moved in the direction (i.e., the direction indicated by the arrow X2) opposite to the direction of the movement of the radiation source 2 during each of the radiation imaging operations.

Further, in FIG. 1, the embodiment of the radiation imaging apparatus for acquiring the chest images is illustrated. The radiation imaging apparatus in accordance with the present invention is also applicable to a mammography apparatus for performing mamma imaging operations. Furthermore, in the embodiment described above, the tomosynthesis imaging operations are performed. The radiation imaging apparatus in accordance with the present invention is also applicable to an apparatus for performing long size imaging operations, such as the radiation imaging operations performed on the entire body of the patient.

Also, in FIG. 2, FIG. 5, FIG. 6, and FIG. 7, various types of the radiation detector moving means 20, 120, 220, and 320 are illustrated. However, the radiation detector moving means is not limited to those described above. It is also possible to employ one of various other known techniques, which are adapted for performing the reciprocal movement of the radiation detector 3, such as a rack-and-pinion technique. Alternatively, a crank mechanism may be employed as the radiation detector moving means 20, and the radiation imaging operation may be performed in the vicinity of the maximum velocity of a trigonometric function-like velocity alteration, which is obtained with the mechanism for converting the rotating movement into the reciprocal movement.

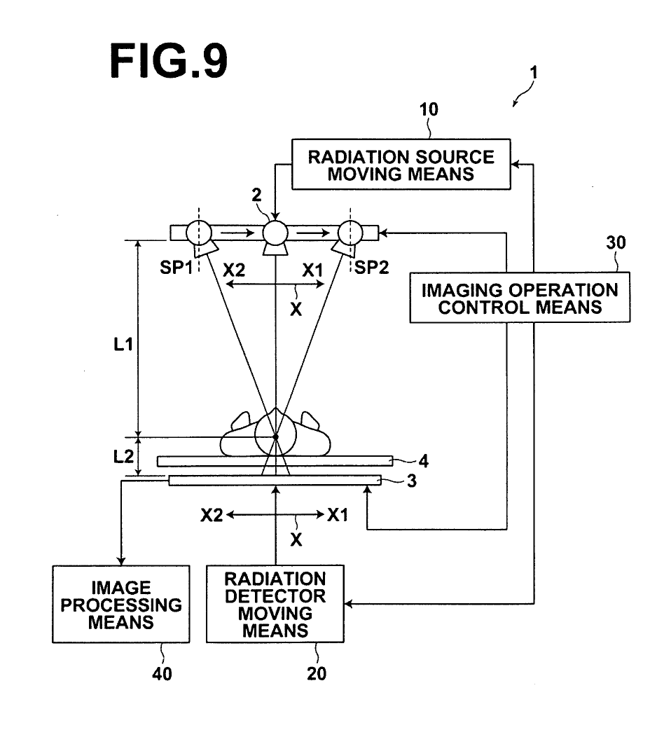

Further, with the radiation imaging apparatus 1 illustrated in FIG. 1, the radiation source 2 and the radiation detector 3 are moved in parallel with the longitudinal direction of the bed 4 (i.e., the direction of the body axis of the patient). Alternatively, as illustrated in FIG. 9, a radiation imaging apparatus 101 may be constituted such that the radiation source 2 and the radiation detector 3 are moved in parallel with the direction (i.e., the direction of the shorter side of the bed 4), which intersects approximately perpendicularly to the body axis of the patient. Specifically, with the radiation imaging apparatus, wherein the size of the movement mechanism for the radiation detector 3 is kept small as described above, it is possible to perform the tomosynthesis imaging operations even with the bed 4 having a small width, wherein the movement distance is limited to a short distance.

Also, in the cases of the radiation imaging apparatus 101 illustrated in FIG. 9, wherein the region, in which the patient body is present, with respect to the direction of the movement of the radiation detector 3 is smaller than in the conventional radiation imaging apparatuses, the irradiation of the radiation to the regions other than the region of interest of the object S in each of the tomosynthesis imaging operations is suppressed, and the radiation dose delivered to the object S is suppressed to the minimum. Specifically, in cases where the tomosynthesis imaging operations are performed, it is necessary that the radiation is irradiated from the radiation source 2 obliquely with respect to the region of interest of the object S. For example, in cases where the tomosynthesis imaging operations are performed on the entire area of the chest acting as the region of interest, the radiation may be irradiated only to the chest with the radiation imaging apparatus 101. However, in the cases illustrated in FIG. 1, the radiation will be irradiated also to the site above the chest and the site under the chest. Specifically, with the radiation imaging apparatus 101 illustrated in FIG. 9, wherein the radiation source 2 and the radiation detector 3 are moved in parallel with the direction, in which the sites other than the region of interest of the patient are not present between the radiation source 2 and the region of interest of the patient, the radiation dose delivered to the patient is kept small.

What is claimed is:

1. A radiation imaging apparatus, comprising:
i) a radiation source for irradiating radiation to an object,
ii) a radiation detector for detecting the radiation, which has been irradiated from the radiation source to the object, and which carries image information of the object, as a radiation image,
iii) radiation source moving means for linearly moving the radiation source along a predetermined direction with respect to the object,
iv) radiation detector moving means for moving the radiation detector in parallel with the direction of the movement of the radiation source, and
v) imaging operation control means for controlling such that a radiation imaging operation, wherein the radiation source irradiates the radiation to the object, while the radiation source is being moved in one direction, is performed a plurality of times with the radiation source taking various different positions,
the imaging operation control means controlling the radiation detector moving means such that the radiation detector is reciprocally moved within a period, during which the radiation source is moved in the one direction from a stage of beginning of the plurality of times of the radiation imaging operations to a stage of finishing of the plurality of times of the radiation imaging operations, and such that the radiation detector is moved in a direction opposite to the one direction of the movement of the radiation source within each of radiation imaging operation periods.

2. A radiation imaging apparatus as defined in claim 1 wherein the imaging operation control means controls such that a movement velocity of the radiation source is approximately identical among the plurality of the radiation imaging operation periods, and such that the movement velocity of the radiation detector is approximately identical among the plurality of the radiation imaging operation periods.

3. A radiation imaging apparatus as defined in claim 1 wherein the imaging operation control means controls such that the radiation imaging operations are performed at predetermined imaging operation intervals, such that the radiation source is moved approximately at a predetermined velocity from an imaging operation beginning position to an imaging operation finishing position, and such that the radiation detector is reciprocally moved approximately with a predetermined period, and
the imaging operation control means controls in accordance with the movement velocity of the radiation source and the period; with which the radiation detector is reciprocally moved, such that the radiation source and the radiation detector are moved in opposite directions within each of the radiation imaging operation periods.

4. A radiation imaging apparatus as defined in claim 3 wherein the imaging operation control means adjusts a timing, with which the driving of the radiation source moving means or the radiation detector moving means is begun, such that the radiation source and the radiation detector are moved in the opposite directions at the time of a first radiation imaging operation.

5. A radiation imaging apparatus as defined in claim 1 wherein the radiation detector moving means is provided with driving means for reciprocally moving the radiation detector with a predetermined period, and
a balancing weight member, which is associated with the driving means, and which is reciprocally moved in a direction opposite to the direction of the movement of the radiation detector.

6. A radiation imaging apparatus as defined in claim 1 wherein the imaging operation control means sets a distance of the movement of the radiation detector within each of the radiation imaging operation periods in accordance with the distance of the movement of the radiation source within each of the radiation imaging operation periods.

7. A radiation imaging apparatus as defined in claim 1 wherein the imaging operation control means calculates a blur quantity on the radiation detector from a distance of the movement of the radiation source within each of the radiation imaging operation periods and moves the radiation detector within each of the radiation imaging operation periods by a distance corresponding to the calculated blur quantity.

8. A radiation imaging apparatus as defined in claim 1 wherein the radiation imaging apparatus further comprises image processing means for forming an image of a predetermined objective plane of the object by use of the plurality of the radiation images having been detected by the radiation detector.

9. A radiation imaging apparatus as defined in claim 1 wherein, at the time at which the plurality of times of the radiation imaging operations are performed on a patient acting as the object, the radiation source and the radiation detector are moved in parallel with the direction, which intersects approximately perpendicularly to a body axis of the patient.
